(19) United States
(12) Patent Application Publication
(10) Pub. No.: US 2012/0209125 A1
(43) Pub. Date: Aug. 16, 2012

(54) Singlet Oxygen Production and Dosimetry for Photodynamic Therapy

(75) Inventors: Steven J. Davis, Londonderry, NH (US); Seonkyong Lee, Boston, MA (US)

(73) Assignee: PHYSICAL SCIENCES, INC., Andover, MA (US)

(21) Appl. No.: 13/370,611

(22) Filed: Feb. 10, 2012

Related U.S. Application Data
(60) Provisional application No. 61/441,548, filed on Feb. 10, 2011.

(51) Int. Cl. A61B 6/00

(52) U.S. Cl. 600/476

(57) ABSTRACT

An apparatus for photodynamic therapy (PDT) includes a light source configured to provide excitation light for a photosensitizer, an optical system configured to direct the excitation light to a target region and receive light emitted by the photosensitizer and/or singlet oxygen generated in the target region, and a detection system configured to receive the light emitted by the photosensitizer and/or the singlet oxygen. The apparatus also includes a filter system configured to spectrally discriminate between emission from the photosensitizer and the singlet oxygen and a processor configured to determine concentrations of the singlet oxygen and/or the photosensitizer based on an emission signal measured by the detection system.

### CROSS-REFERENCE TO RELATED APPLICATIONS

This application claims the benefit of and priority to U.S. provisional patent application No. 61/441,548 filed Feb. 10, 2011, the entire contents of which are incorporated by reference herein.

### GOVERNMENT RIGHTS

The invention was made with government support under the following grants: USAF Contract No. F29601-97-C-0156, NIH Grant No: 1R43CA96243-01, NIH Grant No: 2R44 CA0964243-02, NIH Grant No: R44CA128364-01, and NIH Grant No: 2R44CA119486-04. The government has certain rights in the invention.

### FIELD OF THE INVENTION

This invention relates generally to the production and detection of singlet molecular oxygen produced when light interacts with photosensitizers (both endogenous and exogenous) in tissue. This interaction, often referred to as a photodynamic process, can be exploited in cancer therapies and acne treatments using photodynamic therapy (PDT). The singlet oxygen can cause cancer cell destruction and knowledge of its concentration during the PDT process can be used to understand and optimize the therapy.

### BACKGROUND

Photodynamic therapy (PDT) is a relatively new, rapidly developing, and promising modality for cancer treatment. PDT is a process that uses light and a photosensitizer to produce singlet molecular oxygen. Certain photosensitizers can be preferentially retained in malignant tumors.

When exposed to light, a photosensitizer initiates a reaction that selectively kills the malignant cells to which they are attached. FDA approval has been granted for treatment of esophageal and certain lung cancers. PDT is being used in clinical trials for bladder, brain, skin and other cancers. PDT is also being applied to important areas outside of cancer treatment including age related macular degeneration and actinic keratosis, a pre-cancerous skin condition. Most recently, PDT has been under investigation for treatment of non-cancerous skin conditions such as acne. There is considerable evidence that singlet molecular oxygen (O₂(a¹Δ)) is the active species in cancer cell or endothelial cell necrosis. Despite the general acceptance of this role of singlet oxygen in PDT, there have been limited demonstrations of its importance in vivo. A device that can measure the singlet oxygen during PDT treatments can provide information about PDT dosimetry and the potential of individualized therapeutic design.

Photophrin II was the first widely used photosensitizer (PS). It has strong photodynamic effect, and its major absorption band for photoactivation is at about 630 nm. From a fundamental perspective, 630 nm light does not penetrate tissue as deeply as longer wavelengths. Continuous wave (CW) dye lasers used in PDT are expensive and relatively difficult to operate, and are rapidly being replaced by high power diode lasers that operate in the 630 to 690 nm. There has also been considerable activity to develop photosensitizers with longer wavelength absorption bands to treat tumors at greater depths. They also may be excellent receptors for diode laser excitation.

There has been great interest in developing a sensor for singlet oxygen that can be used as a real-time dosimeter during PDT treatments. Correlations of the singlet oxygen produced with treatment efficacy can be one important use of such a sensor. Some researchers have attempted to develop dosimeters based on the fluorescence intensity of the photosensitizer in the tumor, but photobleaching of the PS precludes this as an accurate method. Some have used small electrodes to measure total oxygen content in tumors during PDT in animal studies, but have demonstrated deoxygenation during treatments. Other researchers have shown that oxygenation of tissue can enhance PDT efficiency. Since singlet oxygen appears to be the active species, a dosimeter for singlet oxygen produced during PDT would be a valuable tool for improving treatment outcomes. In addition, spatially resolved, simultaneous detection and imaging of the PS and singlet oxygen in the tumor would be a valuable tool for developing a better understanding of the PDT mechanisms and better treatment outcomes.

### SUMMARY OF THE INVENTION

The invention, in various embodiments, features a method and apparatus to monitor the weak singlet molecular oxygen optical emission. The invention can be used for monitoring, feedback control, and optimization of photodynamic treatments. In certain embodiments, monitoring the weak singlet molecular oxygen optical emission can be incorporated into a PDT treatment device that includes feedback control.

In some embodiments, an optical excitation source (pulsed or continuous wave), an ultrasensitive photomultiplier tube or other sensitive optical detector, a process for spectral discrimination, and custom software can be used to measure the singlet oxygen luminescence near 1.27 microns. Fiber coupled diode lasers can be used to produce singlet oxygen both in-vitro and in-vivo and have measured the optical signature of the singlet oxygen near-IR emission under numerous conditions. The relative amount of singlet oxygen produced during PDT treatments of tumors on the flanks of mice has been correlated with the post treatment regression of the tumors. The photosensitizer and/or singlet oxygen can be measured when the light source is on (pulsed and CW sources) or after the source is turned off (pulsed source).

A PDT dosimetry system can utilize detection of singlet O₂ emission and PS fluorescence. A low power, pulsed diode laser-based optical method can monitor PDT photoreaction products. CW sources can also be used. Fiber optic cables and/or liquid light guides can introduce the excitation light and collect the near IR light from the PS and singlet oxygen. A pulsed light emitting diode (LED) can also be used as the excitation source for PDT. A diode laser-based, singlet O₂ monitor can enhance the PDT treatment efficacy and can enable physicians to tailor PDT treatment to match different responses of individual patients during PDT.

The technology features, in various embodiments, (1) a diode laser based dosimeter, (2) a process for the delivery of excitation light and collection of near IR emissions with fiber optic or liquid light guides, (3) custom software that provides real-time singlet oxygen data, (4) single diode laser or LED system for PDT excitation and oxygen dosimeter, (5) simultaneous photosensitizer and singlet oxygen detection, and (6) designs of lens systems to optimize singlet oxygen luminescence transmission from distal end of fiber (and liquid light guides (LLG)) through the spectral dispersion system and onto the detectors. It also includes processes, (1) for removing the long wavelength interference from diode laser emission via a diode laser bandpass filter, for automating filter positions, dwell time, and design of 1.2 to 1.3 micron optical filters in front of detectors, (2) for changing the diode laser and LED pulse-lengths and duty cycle to maximize singlet oxygen production, and (3) for modeling to describe the type II PDT process with diode laser sources and systems. With the spatially resolved two-dimensional (2-D) imaging system, both images of the PS fluorescence and singlet oxygen emission are obtained simultaneously and PS and singlet oxygen concentrations from near-IR radiation can be calculated.

A two-dimensional optical system can provide spatially resolved simultaneous imaging of singlet molecular oxygen (¹O₂) phosphorescence and photosensitizer fluorescence produced by the photodynamic process. A spectral discrimination method can differentiate the weak ¹O₂ phosphorescence that peaks near 1.27 μm from PS fluorescence that also occurs in this spectral region. The detection limit of ¹O₂ emission was determined at a concentration of 500 nM benzoporphyrin derivative monoacid (BPD) in tissue-like phantoms, and these signals observed were proportional to the PS fluorescence. Preliminary in vivo images with tumor laden mice indicate that it is possible to obtain simultaneous images of ¹O₂ and PS tissue distribution.

In one aspect, there is a method for monitoring singlet oxygen during photodynamic therapy (PDT). The method includes directing pulsed excitation light to a photosensitizer in a target region, and receiving light emitted by the photosensitizer and/or singlet oxygen generated in the target region. The returning light is filtered to isolate emission from the singlet oxygen. The emission from the singlet oxygen is monitored, and a concentration of the singlet oxygen is determined based on an emission signal measured by a detection system.

In another aspect, there is an apparatus for monitoring during photodynamic therapy (PDT). The apparatus includes a light source configured to provide excitation light for a photosensitizer and an optical system configured to direct the excitation light to a target region and receive light emitted by the photosensitizer and/or singlet oxygen generated in the target region. A detection system is configured to receive the light emitted by the photosensitizer and/or the singlet oxygen. A processor is configured to monitor the emission from the singlet oxygen between pulses of the excitation light and determine a concentration of the singlet oxygen based on an emission signal measured by the detection system.

In still another aspect, there is an apparatus for monitoring during photodynamic therapy (PDT). The apparatus includes means for directing excitation light to a photosensitizer in a target region, means for receiving light emitted by the photosensitizer and/or singlet oxygen generated in the target region, and means for filtering the returning light to isolate emission from the singlet oxygen. The apparatus also includes means for monitoring the emission from the singlet oxygen and means for determining a concentration of the singlet oxygen based on an emission signal measured by a detection system.

In other examples, any of the aspects above, or any apparatus, system or device, or method, process or technique, described herein, can include one or more of the following features. Feedback control from the processor and/or detection system can be used to control the excitation light source. The photosensitizer and/or singlet oxygen can be monitored between pulses of the excitation light (e.g., when using a pulsed source), or while the excitation light source is on (e.g., when using a pulsed or CW source).

In various embodiments, the detection system includes a photomultiplier tube or other optical detector (e.g., an avalanche photodiode, an array of avalanche photodiodes), an imaging camera, a near infrared camera, a linear array, multiple linear arrays, or a combination of the aforementioned. The detection system can be adapted to detect 1.27 micron radiation from singlet oxygen emission. The detection system can include at least one filter to spectrally discriminate between the emission from the photosensitizer and the singlet oxygen.

The pulsed light source can be a low power, pulsed diode laser. The optical waveguide can include at least one optical fiber and/or at least one liquid light guide. A dichroic beamsplitter can be configured to pass the excitation light from the pulsed light source to the optical waveguide and redirect the light returning from the optical waveguide to the detection system.

The PDT treatment can be used in the treatment of cancers (e.g., esophageal, lung, skin, bladder, and brain), in age related macular degeneration, actinic keratosis, and non-cancerous skin conditions such as acne, sebaceous follicle disorders, psoriasis, and eczema.

Other aspects and advantages of the invention will become apparent from the following detailed description, taken in conjunction with the accompanying drawings, illustrating the principles of the invention by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a diagram of the PDT process and shows the excitation of the PS and subsequent production and decay of singlet oxygen.

FIG. 2 is a diagram that shows the strategy for a detection method using a pulsed diode laser or LED.

FIG. 3 shows data from a PS that was excited with a pulsed diode laser. PS and singlet oxygen emission are shown for two media: acetone and water.

FIG. 4A shows an apparatus for photodynamic therapy (PDT).

FIG. 4B shows a configuration for introducing the excitation radiation and collecting the singlet oxygen emission via optical fibers.

FIG. 5 shows the temporal sequencing of the treatment and singlet oxygen dosimeter laser outputs when a separate treatment laser is used for PDT.

FIG. 6 shows an approach for an integrated PDT treatment/dosimeter using a pulsed diode laser. With this method, singlet oxygen can be continuously measured throughout the PDT treatment.

FIG. 7 shows a singlet oxygen 2D imaging system.

FIG. 8 shows a singlet oxygen detection method. (a) Temporal profiles of singlet O₂ phosphorescence at three bandpass filter positions with 1 μM BPD in methanol. (b) Spectral features of singlet O₂ phosphorescence and total emission intensity. (c) The method of the singlet O₂ image process with the three-filter operation (in vitro).

FIG. 9 shows spatially resolved images (10 mM BPD in methanol). (a) Ambient air saturated. (b) Deoxygenated solutions (nitrogen gas purging through the solution). (c) Total BPD fluorescence and singlet O₂ phosphorescence intensities in the area of the interest marked with a square box in the images of 5×5 mm.

FIG. 10 shows a plot of singlet oxygen phosphorescence and BPD photosensitizer fluorescence as a function of BPD concentration in 5% FBS with 5% TTX-100.

FIG. 11 shows images of the BPD fluorescence and singlet oxygen phosphorescence from two tumor-laden mice. (a) 1.0 mg BPD/body kg. (b) 0.5 mg BPD/body kg.

FIG. 12 shows methods for spectrally isolating the singlet oxygen emission from photosensitizer fluorescence and other potential interferences.

FIG. 13 shows a model prediction with longer pulse widths.

FIG. 14 shows an exemplary pulsed mode operation.

FIG. 15 shows in vitro results with an LED as the excitation source.

### DETAILED DESCRIPTION OF THE INVENTION

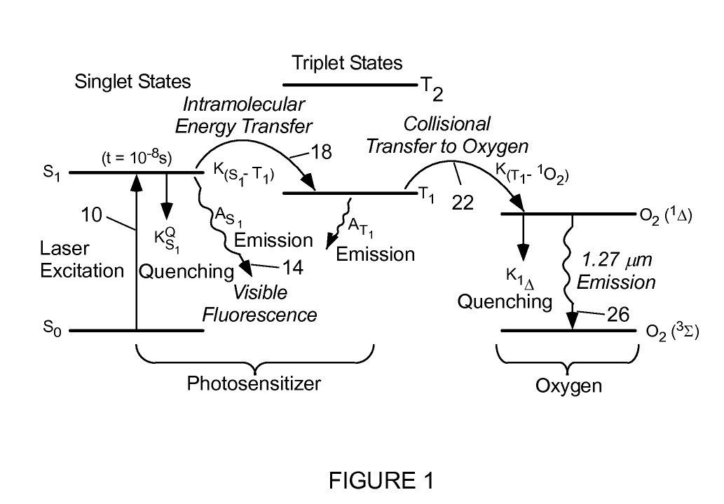

FIG. 1 shows the fundamental type II PDT process. In general, the photosensitizer (PS) absorbs light (10) that excites the PS to the first excited singlet state. The excited singlet state strongly radiates (14) to the ground singlet state emitting optical radiation characteristics of the photosensitizer. Typically, this contains a visible component that can be used to locate the tumor and its boundaries. The excited singlet state also has a large probability of intrasystem crossing (18) to the triplet state. This triplet state is nearly resonant with the transition of oxygen from ground state to excited singlet state. Collisions between this metastable dye molecule and ground state oxygen (present in the tumor) populate the singlet delta state of oxygen via an energy transfer process (22). The singlet oxygen, believed to be the major active species in PDT, emits (26) very weakly in the near infrared near 1.27 microns, and this luminescence can be monitored using a singlet oxygen detection system.

For example, the weak but unique spectral signature of the O₂(a¹Δ [symbol] X³Σ) transition shown in FIG. 1 can be monitored. The radiative emission from the singlet oxygen is extremely weak (radiative rate of 0.2 s⁻¹ in aqueous media such as tissue) and occurs at a wavelength, λ of 1.27 microns, which is in a particularly challenging region of the spectrum for sensitive detection. Indeed, the weak optical emission of singlet oxygen is one of the major difficulties in monitoring singlet oxygen produced by irradiated photosensitizers. While optical filtering provides some measure of sensitivity, temporal discrimination can also be used.

Referring to FIG. 1, the prompt dye fluorescence from the ¹Sₒ state has a lifetime of about 10 ns since it is from a radiatively allowed transition. It decays much more rapidly than the emissions from the singlet oxygen (lifetime of 4 μs in aqueous media and as short as 0.1 μs in biological media). Until recently, the most sensitive optical sensors for singlet emission were solid state, liquid nitrogen cooled germanium photodiode detectors. While these devices can provide high sensitivity (D*−10¹⁵ cm² Hz^{1/2}/W), they operate at what is known as the “gain/bandwidth limit” and the highest sensitivity Ge devices have a temporal response time of 1 ms. This is inadequate for isolating the singlet oxygen emission from prompt dye emission that may leak through to the detector. The detector simply cannot discriminate between laser on and laser off conditions with adequate temporal resolution.

A pulsed diode laser can be integrated with a near IR photomultiplier tube (PMT) with a time response<5 ns. This PMT has low enough dark current so that photon counting methods can be used to optimize the sensitivity. Diode lasers can produce pulses of continuously tunable pulse-lengths from a few nanoseconds to CW operation. Unlike Q-switched Nd:YAG lasers (pulse-lengths of about 10 ns), a relatively long (about 5 μs) diode laser pulse does not produce significant energy compression. While the diode laser is on, its peak power is essentially equal to that when operating CW. Consequently, no direct tissue damage typically results. For example, for a 300 mW diode laser, pulses of 2 μs duration contain only 0.6 0μJ, and each pulse has a peak power of 300 mW and can cause no tissue damage.

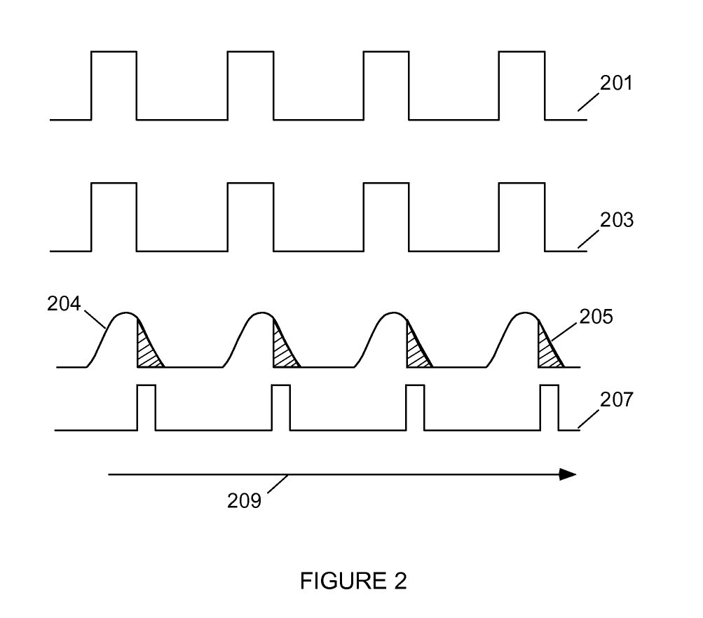

FIG. 2 shows a detection strategy using a pulsed diode laser or LED excitation source. Waveform 201 is the waveform of the pulsed excitation source. Waveform 203 is the waveform of the fluorescence from the photosensitizer. Waveform 204 is the waveform of the singlet oxygen luminescence produced by pulsed PDT source. The shaded area 205 shows singlet oxygen signal after light excitation pulse shuts off. Waveform 207 is the waveform of the gated detection system, where the singlet oxygen is detected. The axis 209 is the time axis. The singlet state of the PS decays in about 10 ns, and the singlet oxygen emission has a lifetime of about 100-200 ns in tissue. Thus, temporal discrimination can be used to monitor the singlet oxygen emission while the diode laser is off.

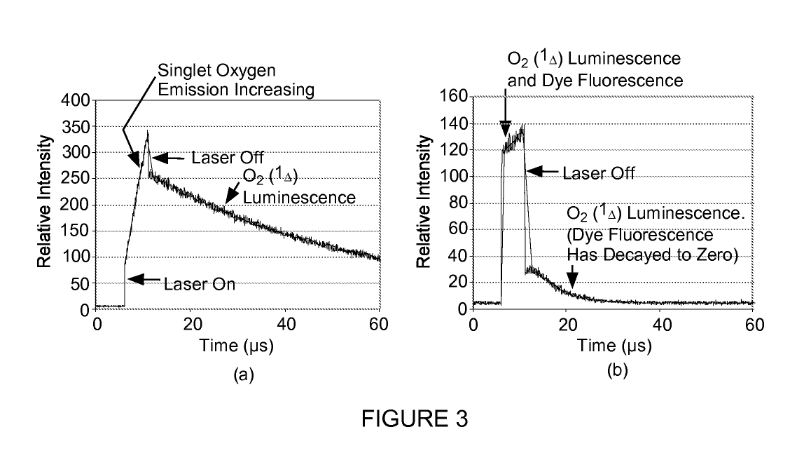

FIG. 3 shows typical signals recorded with a near-IR PMT. FIGS. 3(a) and 3(b) show data for the PS Cl-e6 in acetone (FIG. 3a) and water (FIG. 3b) for a 5 μs diode laser excitation pulse width. The temporal evolution of the production of O₂(¹Δ) (via transfer from the photosensitizer triplet state) and its subsequent quenching (by the solvent molecules) are evident in these data. During the square wave diode laser pulse, the singlet oxygen signal grows in both the acetone and water solvents via the energy transfer process discussed above (see FIG. 1). For the acetone solution (FIG. 3a), the quenching is relatively weak and the singlet oxygen emission by the end of the diode laser pulse is several times stronger than the near-IR fluorescence from the PS. In contrast, for the more severe quenching aqueous environment, the singlet oxygen emission is much weaker. Note also the dramatic reduction in τ_{Δ} due to water quenching when compared to acetone, a relatively weak quencher of singlet oxygen. These observations show that the singlet oxygen production can be optimized by varying the excitation pulse-length depending upon the quenching environment.

The temporal evolution shown in FIG. 3b is typical of the singlet oxygen signatures that were observed in-vivo. Most of the bright dye fluorescence promptly terminates at the end of the diode laser pulse. The intensity (photoelectron counts) can be summed after the diode laser is shut off to obtain a singlet oxygen signal. However, in tissue, the singlet oxygen becomes so highly quenched that some weak emitters can cause spectral interferences, even when observing during the time that the diode laser is off. The relatively slow emission (phosphorescence and/or luminescence) by some triplet state of the photosensitizer is a potential interference for in-vivo studies. The triplet state lifetime is typically on the order of microseconds and the emission (albeit weak) can occur subsequent to the diode laser pulse. This requires additional optical filtering to isolate the singlet oxygen spectral feature from the broadband emission from the dye. Narrowband filters (e.g., a series of three narrow band interference filters with center wavelengths of 1.22, 1.27, and 1.315 microns) can be used to spectrally discriminate between the photosensitizer and singlet oxygen emission for the in-vivo studies. With this approach, singlet oxygen production from two photosensitizers in tumors implanted in rats and from healthy human skin containing topical ALA photosensitizer were detected.

Diode laser radiation can be delivered and singlet oxygen emission can be collected with fiber optic cables and/or liquid light guides. Fiber delivery and collection systems are compatible with clinical PDT applications.

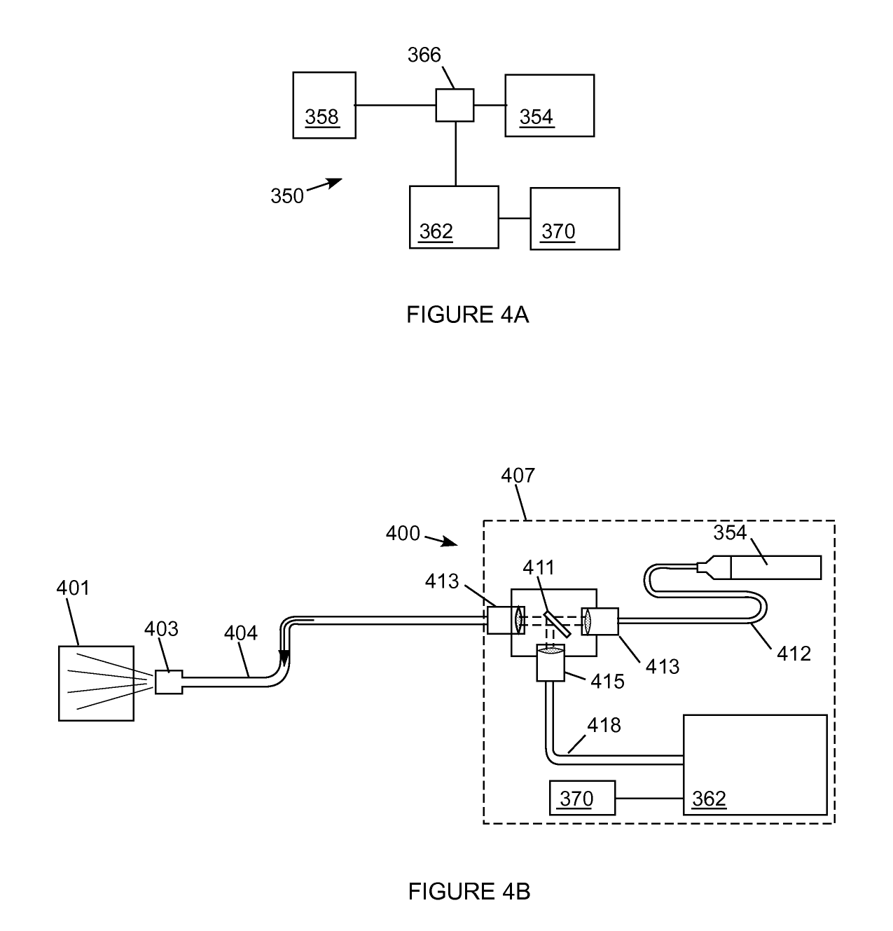

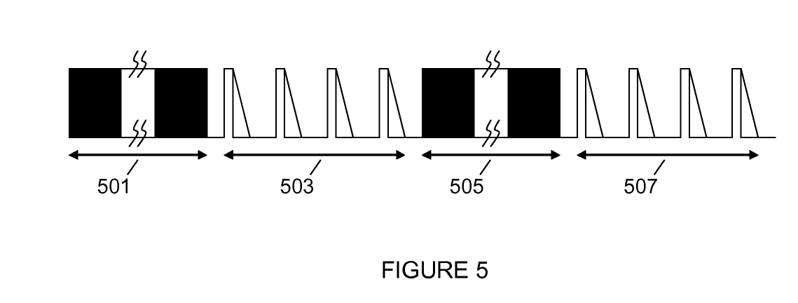

FIG. 4A shows an apparatus 350 for photodynamic therapy (PDT). The apparatus 350 includes a light source 354 configured to provide excitation light for a photosensitizer and an optical system 358 configured to direct the excitation light to a target region and receive light emitted by the photosensitizer and/or singlet oxygen generated in the target region. The apparatus 350 includes a detection system 362 configured to receive the light emitted by the photosensitizer and/or the singlet oxygen. A filter system 366 is configured to spectrally discriminate between emission from the photosensitizer and the singlet oxygen. A processor 370 is configured to determine concentrations of the singlet oxygen and/or the photosensitizer based on an emission signal measured by the detection system.

FIG. 4B shows a configuration 400 for introducing the excitation radiation and collecting the singlet oxygen emission via optical fibers. A sample 401 (in-vitro cell or in-vivo tissue) is illumination. An optic or optical system including a handpiece 403 and an optical fiber 404 directs the excitation light beam and collects the singlet oxygen and/or photosensitizer emissions. The base unit 407 can be a cabinet. The source 354 can be a diode laser, although other sources can be used. A dichroic mirror or interference filter 411 can pass light from the source 354 to the optical fiber 404 and handpiece 403 for delivery to the sample 401. Light returning from the sample 401 is directed to a detection system 362 (e.g., a near-IR photomultiplier). A processor 370 is configured to determine concentrations of the singlet oxygen and/or the photosensitizer based on an emission signal measured by the detection system 362.

The source 354 delivers radiation to the dichroic mirror or interference filter 411 via an optical fiber 412 and fiber optic collimator 413. Optical fiber 404 also includes a fiber optic collimator 413. Light returning from the sample 401 is directed to the detection system 362 via a fiber optic collimator 415 and optical fiber 418. The collimator 415 can include a narrow bandpass filters.

The detection system 362 includes a photomultiplier tube or other optical detector (e.g., an avalanche photodiode, an array of avalanche photodiodes), an imaging camera, a CCD camera, a near infrared camera, a linear array, multiple linear arrays, or a combination of the aforementioned. The detection system 362 can be adapted to detect 1.27 micron radiation from singlet oxygen emission.

The dichroic mirror or interference filter 411 can be a thin pellicle dielectric mirror transmits the diode laser radiation and reflects the 1.27 micron emission from the singlet oxygen. Any diode laser light (630-690 nm) that is reflected or scattered into the detection fiber arm is removed with long pass and narrow band pass filters. This device can be used for both in-vitro and in-vivo studies.

Optical fiber 404 can be a single fiber optic cable containing a central fiber to carry the excitation light to the sample and six fibers that surround the central fiber in a close pack arrangement. The six fibers collect the singlet oxygen emission and transport it to the optical filters that are placed in front of the detection system. This multi-fiber configuration also uses a narrow band filter to remove any out of band, near-IR emission from the diode laser beam. This device can be used for both in-vitro and in-vivo studies. A narrow band pass filter in the diode laser optical path can be used to remove any long wavelength “spontaneous” emission that can produce background interference in at the singlet oxygen emission wavelength near 1.27 microns.

The processor 370 can use a mathematical model of the PDT process using diode laser excitation can be used to predict the observed signals for in-vitro studies. This can be extended to in-vivo conditions and can provide a valuable tool for designing optimal PDT strategies. For example, by varying the pulse length and duty cycle, the singlet oxygen being produced in the PDT process can be optimized.

The processor 370 can perform real-time data analysis of the observed signals, as part of the dosimetry system. Customized software can be used in the instrument for real-time data analysis, which allows the instrument to display the singlet oxygen concentration during the PDT process. The concentration of the photosensitizer can be displayed based on PMT detection.

The interference filter can be a continuously variable liquid crystal filter and/or a fixed wavelength interference filter(s) to spectrally resolve the singlet oxygen luminescence from the PS fluorescence. Additional sensitivity for the instrument can be gained using the filter system(s).

The detection system 362 can include a visible wavelength sensitive CCD camera in conjunction with a near-IR detection system (either a PMT or near-IR camera) to perform simultaneous detection of the photosensitizer and the singlet oxygen emission.

The processor 370 can utilize image reconstruction software to enhance the PS and singlet oxygen spatial images. A near IR camera and/or near IR PMT can be used to determine the PS concentrations directly using the spectral tail process. This can eliminate the need for an alternate visible wavelength PMT. The near IR camera and/or near IR PMT can perform this function as well as measure the singlet oxygen.

The source 354 can combine two diode lasers in an integrated PDT treatment and dosimeter instrument. The diode lasers can be a commercial continuous wave (CW) laser as the PDT treatment laser and a pulsed diode laser for the PS excitation and subsequent singlet O₂ detection.

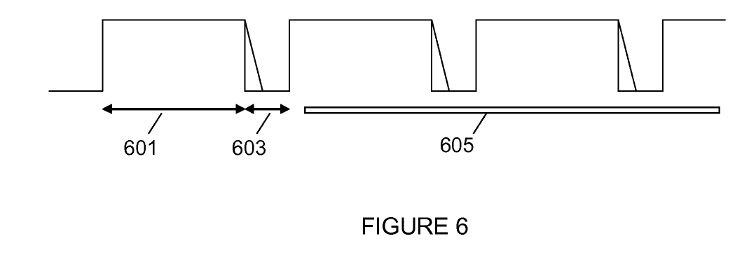

FIG. 5 shows the temporal relationship between the CW treatment laser and the pulsed diagnostic laser. The CW treatment laser irradiates a selected area on tissue, usually containing a tumor model. This irradiation continues for a selected time. Regions 501 and 505 shows “on-time” for conventional continuous wave PDT treatment laser. Then, the CW treatment laser is turned off, and the pulsed diode laser that operates at the same wavelength as the treatment laser is directed at the area that was treated by the CW laser. Typically, the CW laser is on for several minutes and the pulsed laser is on for a few tens of seconds. Pulse train 503 and 507 is the train for a pulsed diode laser for determining singlet oxygen while treatment laser is off.

In this configuration, the pulsed laser produces singlet O₂ that is detected in the area that has just been illuminated by the CW treatment laser. Singlet oxygen emission is monitored, which is proportional to the product of [O₂]×[PS]. Although tissue that was irradiated by the treatment laser can be examined, singlet O₂ need not be monitored during the actual PDT treatment (while the treatment laser is on).

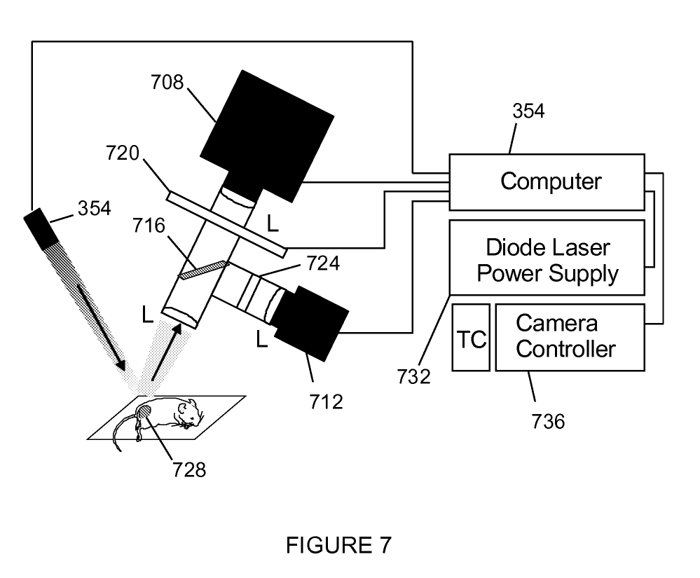

A single laser system with a fiber coupled diode laser can be used as both the treatment and singlet O₂ dosimeter laser for real-time singlet O₂ monitoring (e.g., for in vivo studies). This eliminates the need for a separate CW treatment laser and improves the signal to noise ratio. This configuration is shown in FIG. 6. A single laser system can be used to optimize a maximum singlet O₂ production related to [PS] and [O₂] by varying the light dosage.

Region 601 shows pulse time profile for system where one laser is used for both PDT treatment and singlet oxygen dosimeter. Window 603 is the time window at end of the pulse used to detect singlet oxygen where interference from prompt PS fluorescence is minimal. Region 605 shows several pulses as described in 601 and 603.

A single, pulsed laser can be used to monitor the singlet O₂ produced by each “treatment” laser pulse. By varying the light dosage in several media including methanol, water, and intralipid (IL) solutions, the optimization of the singlet O₂ production can be investigated. The duty cycle of the pulsed laser was varied from 1% to 25%, although larger or smaller duty cycles can be used depending on the application. The maximum singlet O₂ production was observed with the pulsed laser of about 3 μJ/pulse (about 10% duty cycle) with BPD in 5% IL solutions.

The maximum signal of singlet O₂ production was observed to occur at different duty cycles with the pulsed system depending upon the PS environment. Indeed, PDT treatments depend upon photosensitizer concentration and bleaching by the laser in addition to the relative concentration of oxygen in the volume being treated. This capability offers potential benefits of the treatment optimization. This configuration of the single, pulsed diode laser for both PDT treatment and singlet O₂ detection allows one to investigate the real time PDT treatment response by varying light dosages for a better treatment outcome.

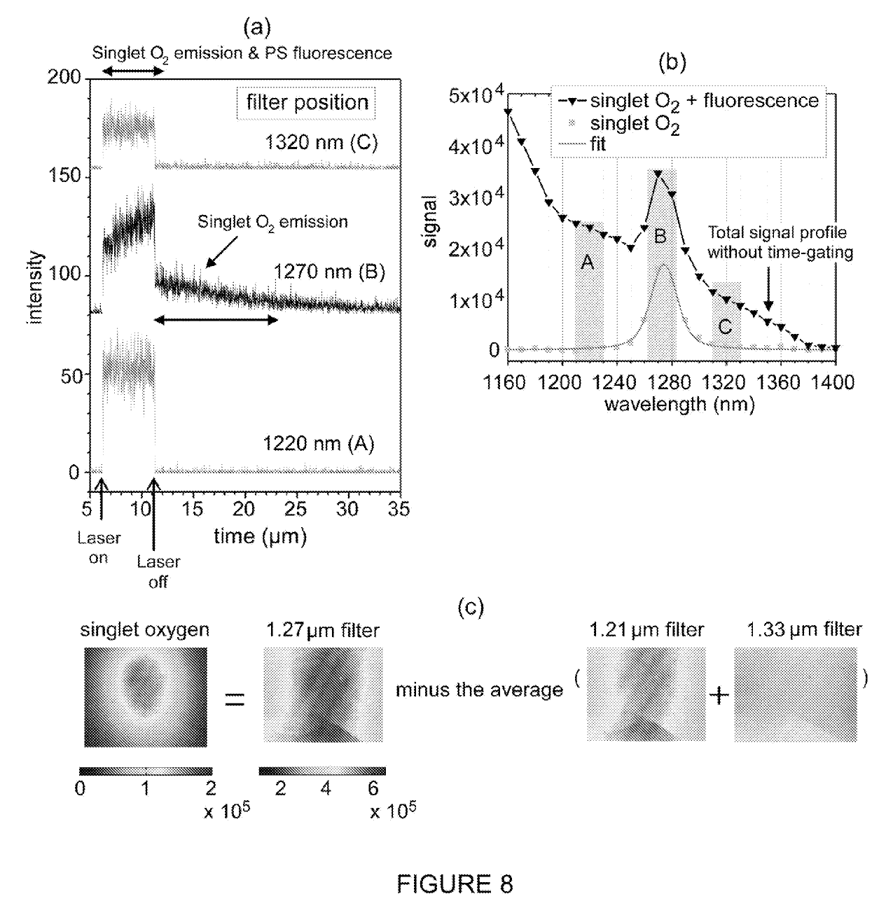

FIG. 7 shows a schematic of a 2D imaging system 700 including of an excitation source 354, near-IR sensitive camera 708, and visible wavelength sensitive camera 712. The imaging system is capable of simultaneous registration of images of ¹O₂ phosphorescence and PS fluorescence in a time frame of a few minutes, compared with typical raster-scanning methods that take tens of minutes to map out an entire area of interest in order to achieve a similar spatial resolution of 25-50 μm in the near-IR spectral region. Fast image acquisition becomes critical because the PDT treatment needs to be monitored in real-time without compromising the detection quality in a clinical environment.

The imaging system 700 includes a beam splitter 716 to pass radiation to the near-IR camera 708 and direct radiation to the visible wavelength sensitive camera 712. Near-IR camera 708 has a filter 720 and visible camera 712 has a filter 724. The system is being used to observe a tumor 728 on a mouse, although a system can be configured for in vitro studies. The imaging system 700 includes a processor 370, a power supply 732 for the source 354, and a controller 736 for the cameras. Filter 720 can be a filter set that is controlled by the processor 370.

The near-IR camera 708 (MOSIR 950, 26.6×6.7 mm, 1024×256 pixels) can be used for the ¹O₂ phosphorescence detection. This camera uses a high quantum efficiency photocathode and an electron bombardment intensifier to provide near single-photon detection in the 1 to 1.5 μm spectral region. The visible camera 712 (Pike F-145, 9.0×6.7 mm, 1392×1040 pixels) can be used for the visible PS fluorescence measurement. The focal length of the dual beam imaging system was 55 mm, and collimated light from the image area was split between the IR camera and the visible camera through a beam-splitter.

For the BPD excitation, a fiber coupled diode laser with the wavelength centered at 692 nm (˜130 mW/cm²) was operated at a repetition rate of 10 kHz with a pulse width of 5 μs. The beam size of the excitation laser was 15 mm in diameter at the focal plane of the imager, and an optical diffuser was used to generate uniform excitation spot. A time-gating rate of the near-IR camera is insufficient for rapid data accumulation. A non-gating mode (continuous mode) for the near-IR camera with appropriate spectral background subtraction can be used. In the non-gating mode, the camera is focused on the fluorescence volume and determines the amounts of photoelectrons for a preset length of time.

For the ¹O₂ detection, three spectral images were recorded in rapid succession using a computer controlled slider 720 containing three bandpass (BP) filters centered at 1.22, 1.27 and 1.32 μm with a full width at half maximum (FWHM) bandwidth of 15 nm. These filters were used to spectrally isolate the ¹O₂ emission near 1.27 μm from the long wavelength spectral background signal, such as PS fluorescence and/or phosphorescence, and autofluorescence.

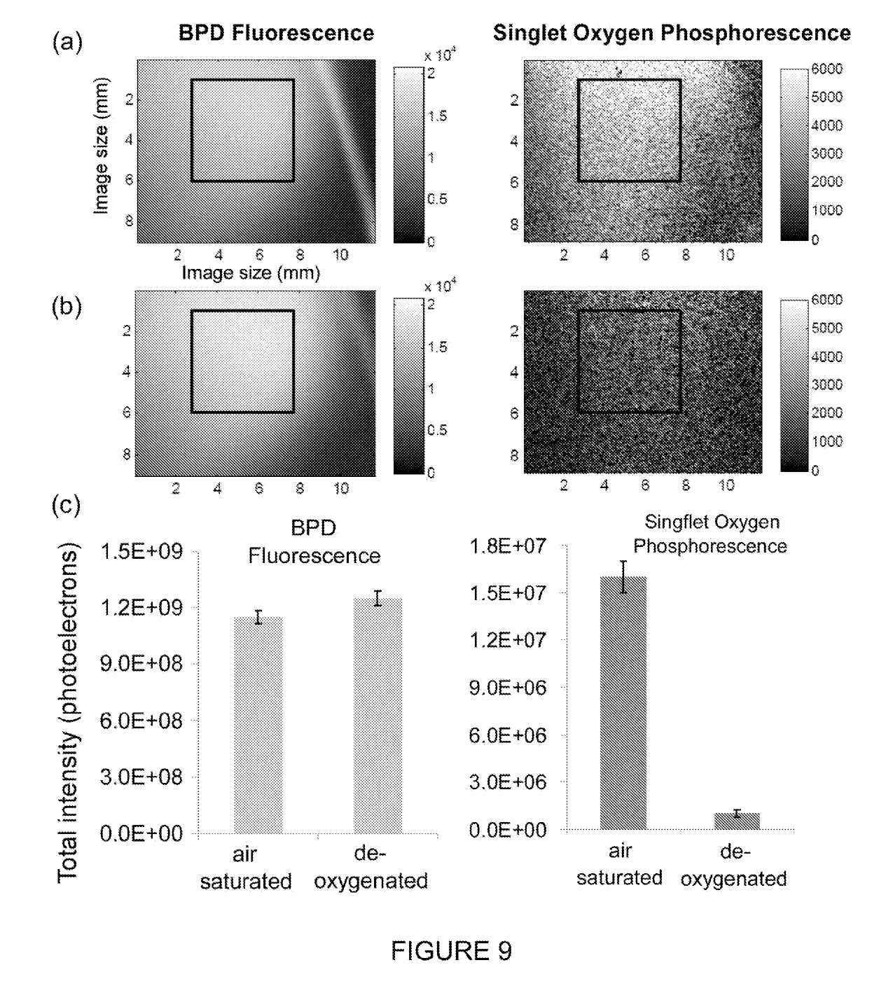

The emissions at 1.22 and 1.32 μm (out-of-the band wavelengths) contain only PS fluorescence while the emission at 1.27 μm contains contributions from both the ¹O₂ and PS, as shown in FIG. 8A obtained in-vitro using BPD (1 μM in methanol). The images recorded at 1.22 and 1.32 μm were co-registered and averaged to generate a single spectral image for PS fluorescence. This formed a first order average of the signal level of the PS fluorescence contribution at 1.27 μm image. This averaged PS fluorescence image was subsequently subtracted on a pixel by pixel basis from the image obtained with the 1.27 μm filter. For the visible PS fluorescence detection, a BP filter was selected to transmit specific wavelength region for the PS fluorescence to the CCD camera. Each bandpass filter was selected for a particular PS to optimize the transmission and spectral discrimination.

To investigate the background signal level further around the ¹O₂ emission band, a liquid crystal tunable filter (Cambridge Research & Instrumentation, Inc., model# LNIR-06, FWHM=6 nm) was used to obtain a detailed spectral profile around 1.27 μm. FIG. 8B shows how the long wavelength (1.2-1.4 μm) PS fluorescence is recorded and subtracted from the entire PDT emission spectrum to provide the emission due to the ¹O₂. In the current 2D imaging system, both ¹O₂ phosphorescence and PS fluorescence were collected, as shown in the upper trace of the triangle symbols in FIG. 8B with three optical filters centered at 1.22, 1.27 and 1.32 μm. The shaded areas under A, B, and C in FIG. 8B represent the total light intensities that were measured for out-of-the band baseline signals (A and C) and in-band signal of ¹O₂ intensity and baseline contribution (B). By subtracting the average baseline signal (average value of A and C) from the signal B, the ¹O₂ intensity was calculated. This spectral discrimination approach is essential to distinguish the ¹O₂ emission from other long wavelength background signals, as mentioned above. As shown in FIG. 8B, a single long-pass filter with a 1.27 μm filter is not able to robustly differentiate ¹O₂ phosphorescence from other long wavelength background signals. This is especially true for in vivo measurements where the ¹O₂ phosphorescence is extremely weak relative to the underlying fluorescence background.

FIG. 8C shows an example of how the images of ¹O₂ phosphorescence were acquired. For acquisition of the ¹O₂ image data, the diode laser beam was directed onto the face of a 1 cm square cuvet which contained 50 μM chlorine e6 (Cl-e6) in phosphate buffer solution. The pixel by pixel averaged values of the 1.22 and 1.32 μm images was subtracted from the image recorded at 1.27 μm to produce the image of the ¹O₂ phosphorescence.

The spatial resolutions of both the visible and near-IR imaging systems were measured using a standard Air Force test pattern. This imaging system was developed to image the entire area of light illumination, ˜1×1 cm, and the magnification of the imaging system was optimized to image the entire area to the detector. The respective spatial resolutions for the visible and near-IR systems were <50 μm and <100 μm estimated based on a FWHM limit of a line spread function method.

¹O₂ phosphorescence imaging from BPD in methanol, water, FBS, and intralipid solutions was investigated. These solvents were used to provide a variety of quenching environments. BPD solutions were procured from U.S. Pharmacopeia (Verteporfin) and solvents from Fisher Scientific. BPD concentrations covering the range 10⁻⁴ to 10⁻⁶ molar were prepared. All mixed BPD solutions were kept in amber glass bottles to minimize any interactions with room lights.

A preliminary study of ¹O₂ production during PDT in tumor laden mice was also conducted. The BPD photosensitizer is commonly used for treatment of age related macular degeneration, and has been initiated in studies for solid pancreas tumors. The ¹O₂ generation of BPD is not as well studied as Cl-e6 or 6-aminoleuvulinic acid-induced protoporphyrin IX (ALA-induced PpIX), but this is more commercially available drug. All animal procedures were carried out according to protocols approved by the Dartmouth College Institutional Animal Care and Use Committee (IACUC). Pancreatic tumor cells were implanted subcutaneously in 6-week-old male nude mice (˜22 g).

AsPC-1 cells, derived from a human pancreatic acinar cell adenocarcinoma (CRL-1682, American Type Culture Collection (ATCC), Manassas, Va. 20108) were cultured in RPMI 1640 with 10% (v/v) fetal bovine serum (FBS), 1% (v/v) penicillin-streptomycin prepared for a stock solution of 10,000 IU penicillin and 10,000 g/ml streptomycin (Mediatech Herndon, Va.), 2 mM L-glutamine, 10 mM HEPES, 1 mM sodium pyruvate, 4500 mg/L glucose, and 1500 mg/L sodium bicarbonate. The cells were passed by washing twice with phosphate buffer solution (PBS) without calcium and magnesium and then incubated at 37° C. with 0.25% trypsin for 5-10 minutes. When all the cells had lifted off from the bottom of the culture flask, the trypsin was neutralized with culture medium and the cell solution was pelleted and cells suspended in complete medium at 4×10⁷ cells/ml.

The cells, required for implantation, were prepared in a 1:1 mixture of cell culture medium and Matrigel® (BD Biosciences, San Jose, Calif.). Matrigel® was thawed on ice in a 4° C. refrigerator overnight and was kept on ice for the entire implantation procedure. AsPC-1 cells were diluted in a 1:1 ratio of culture medium and Matrigel® to a final concentration of 4×10⁷ cells/ml for implantation. Sterile insulin syringes (12 cc U-100 Lo-Dose Insulin Syringe 281/2, Becton Dickinson & Co., Franklin Lakes, N.J.) were loaded with the cell-Matrigel® solution and placed and kept on ice ready for the implantation procedure.

Once the mouse was sedated using isoflurane gas (O₂ flow meter set to 1 L/min; induction at 3% then reduced to 1.5-2%), the left side of the mouse's abdomen was sterilized with an iodine solution (Povidone-Iodine, Novaplus, Irving, Tex.) and the cell-Matrigel® solution (1×10⁶ cell in 50 μl) was injected subcutaneously. The Matrigel® was allowed to set (˜10 seconds) and the needle was gently removed from the injection site and swabbed with iodine to kill any stray cells in the injection site. The growth of the AsPC-1 tumors in each mouse was studied two weeks after implantation so that an average tumor volume of approximately 90 mm³ was reached for the in vivo BPD study.

BPD doses of 0.5, 1, and 2 mg/body weight kg were used for this part of the study. Verteporfin for injection was obtained from QLT Inc. (Vancouver, Canada) as a gift. Verteporfin for injection is composed of a sterile liposomal formulation of BPD-MA (Visudyne, Novartis, N.Y.). A stock saline solution of Verteporfin was reconstituted in water according to the manufacturer's guidelines, using 2.5% as the active component. Animals were injected intravenously, via the lateral tail vein, with 75 ul of Verteporfin to achieve the required dose of 0.5, 1 or 2 mg/kg body weight.

After one hour to allow for systemic tissue distribution and uptake within the tissue organs, the mouse was anesthetized for in vivo imaging. Gas anesthesia is the preferred method of immobilization for in vivo imaging of mice and rats and isoflurane gas is minimally metabolized (<0.17%) by the liver and therefore is less toxic to the animal's metabolism as compared to injectable anesthetics. Once the mouse was sedated, the skin around the tumor was carefully cut and drawn back to expose the tumor tissue situated subcutaneously. The mouse was transferred to the imaging platform of the dual-channel imaging system and placed in position so that its nose was in front of the nose cone attached to the isoflurane anesthesia system. The imaging system platform has an electric heat pad integrated in order to maintain animals warm during anesthesia in order to prevent hypothermia. Once the ideal position had been achieved, images of BPD fluorescence and ¹O₂ phosphorescence were acquired using the visible and near-IR cameras respectively. Each mouse took approximately 10 minutes to image. After the tumor side was imaged, the skin on the contralateral side of the mouse was removed and normal tissue was imaged for comparison. Following the completion of imaging, the anesthetized mouse was euthanized by cervical dislocation.

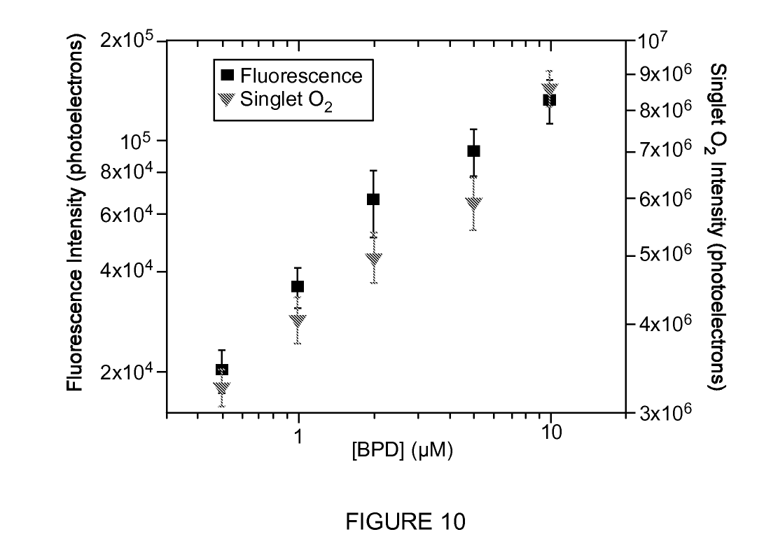

To characterize the imaging system, a series of in vitro studies were conducted with the BPD photosensitizer in several media including protein-laden aqueous solutions that are severe quenchers of ¹O₂. FIG. 9A shows the spatially resolved images of both the ¹O₂ phosphorescence (right panel) and BPD fluorescence (left panel) in methanol recorded for 10 seconds through each of the three optical filters. To verify that this signal originates from ¹O₂, nitrogen gas was bubbled through the sample bottle to displace the dissolved oxygen. When the solution was deoxygenated as shown in FIG. 9B, the ¹O₂ phosphorescence signal essentially disappeared while the PS fluorescence increased slightly. The intensities of the ¹O₂ phosphorescence and BPD fluorescence were calculated within the illumination areas as shown in FIG. 9C. The total photoelectron counts from the ¹O₂ phosphorescence decreased more than 90% when the sample was deoxygenated. However, the PS fluorescence was observed both with oxygenated and deoxygenated conditions because PS fluorescence is independent of the oxygen concentration in the solution. The slight enhancement of the PS fluorescence in the deoxygenated sample may be due to the evaporation of the methanol solvent during the deoxygenation process resulting in a little higher PS concentration.

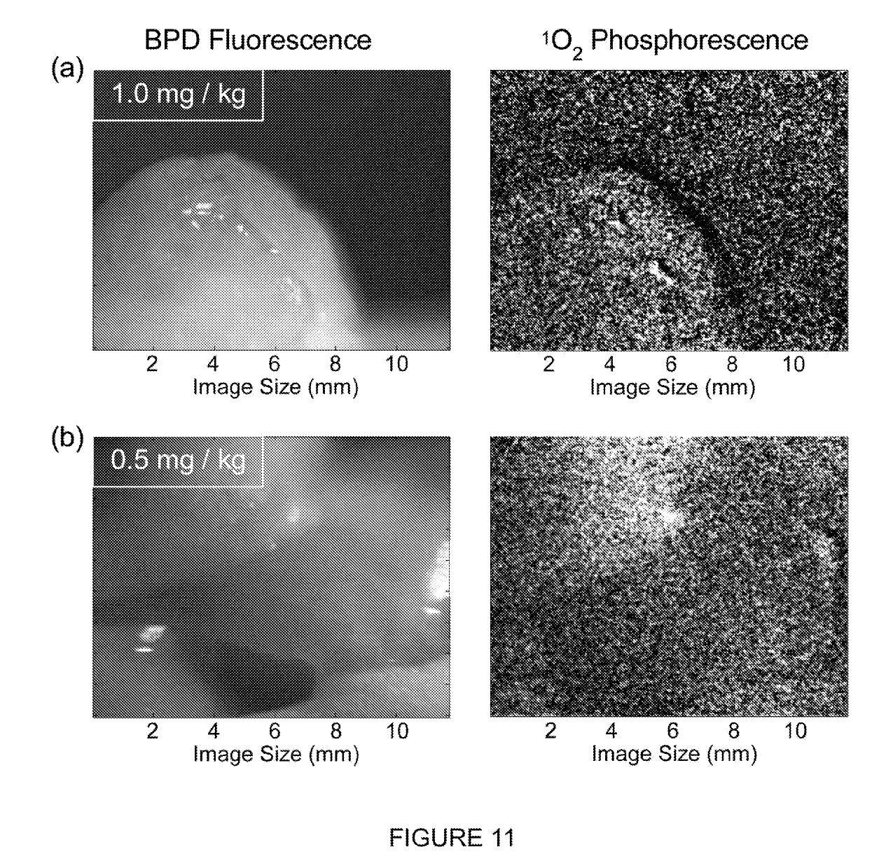

FIG. 10 shows the plot of both the ¹O₂ phosphorescence and BPD fluorescence intensities (from spatially resolved images) as functions of the BPD concentration in a highly quenching FBS environment. These data were obtained in 40-50 seconds at each optical filter position to increase signal-to-noise level. There is a strong correlation between the PDT produced ¹O₂ and the PS fluorescence. Spatially resolved images of the ¹O₂ phosphorescence and the PS fluorescence were obtained with the BPD concentration as low as 500 nM in FBS solution as well as in a highly scattering environment using 2-5% intralipid solution.

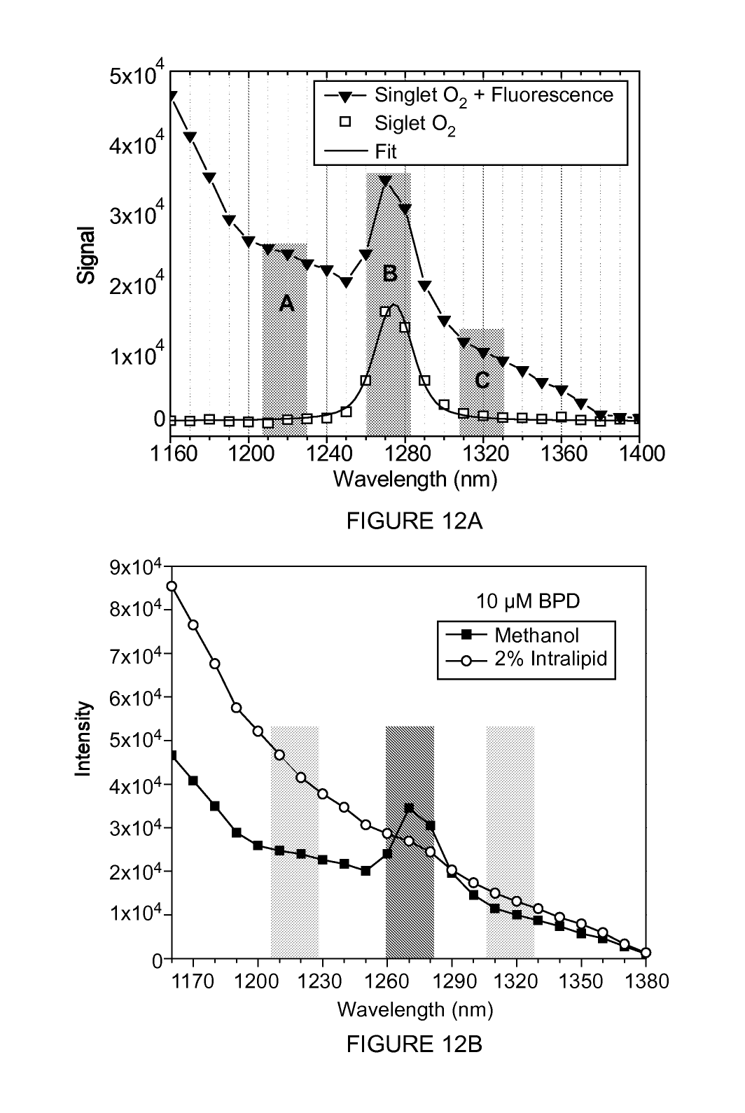

Simultaneous images of the spatial locations were recorded for both the BPD fluorescence and ¹O₂ phosphorescence, as shown in FIG. 11, for two mice with tumors implanted as described above. These images were recorded with skin removed tumor sites one hour after BPD injection. The BPD photosensitizer accumulation in the AsPc1 pancreatic model has been a challenge for these preliminary experiments because of the lack of extensive vascular structure and considerable stroma associated with this tumor model. Images that clearly show a strong correlation between the BPD fluorescence and ¹O₂ phosphorescence have been obtained using image data reduction algorithms. Some of the spatial features are common in both the PS and ¹O₂ images. In addition, the uniformities of the intensities for the two species differ indicating that the ¹O₂ and PS spatial profiles are distinct. In principle, it is possible to determine the concentrations of accumulated/photobleached PS and ¹O₂ production in the tumor sites from these images. Monitoring spatially resolved images of both PS and ¹O₂ can be a good indication of the PDT treatment process whether the PDT efficacy is limited by oxygen availability or the localized tissue PS concentration.

Data processing can include a temporal spectrum for each wavelength measurement point, background signal calculation using out-of-band wavelength measurement points, and signal processing of in-band of singlet O₂ emission. The temporal spectrum for each wavelength measurement point can include a dark baseline subtraction of a light source (e.g. diode laser or a LED) OFF region from a light source ON region, and time-gating under singlet O₂ lifetime.

Background signal calculation using out-of-band wavelength measurement points utilizes spectral features of out-of-band and in-band regions of singlet O₂ emission band (centered at 1270 nm). Unique features of the out-of-band background in near-IR depend on environment where singlet O₂ is produced such as solvents, tissue type, and media tested. Linear or non-linear expression of the out-of-band structure with several wavelength points outside singlet O₂ emission band and out-of-band background library with specific sets of functions (combination of basis functions). The background signal components include the long wavelength tail of photosensitizer fluorescence, autofluorescence and systematic instrumental baseline.

Signal processing of In-band of singlet O₂ emission incovles subtracting underlying background signal component from in-band singlet O₂ signal. Linear estimation of background is a three-filters approach including signal at singlet O₂ band (ex. 1270 nm optical filter) and averaged signal at two out-of-band filters (e.g. 1220, 1320 nm optical filters). Non-linear estimation of background is a multiple wavelength approach including a curve fitting method with out-of-band wavelength measurement points.

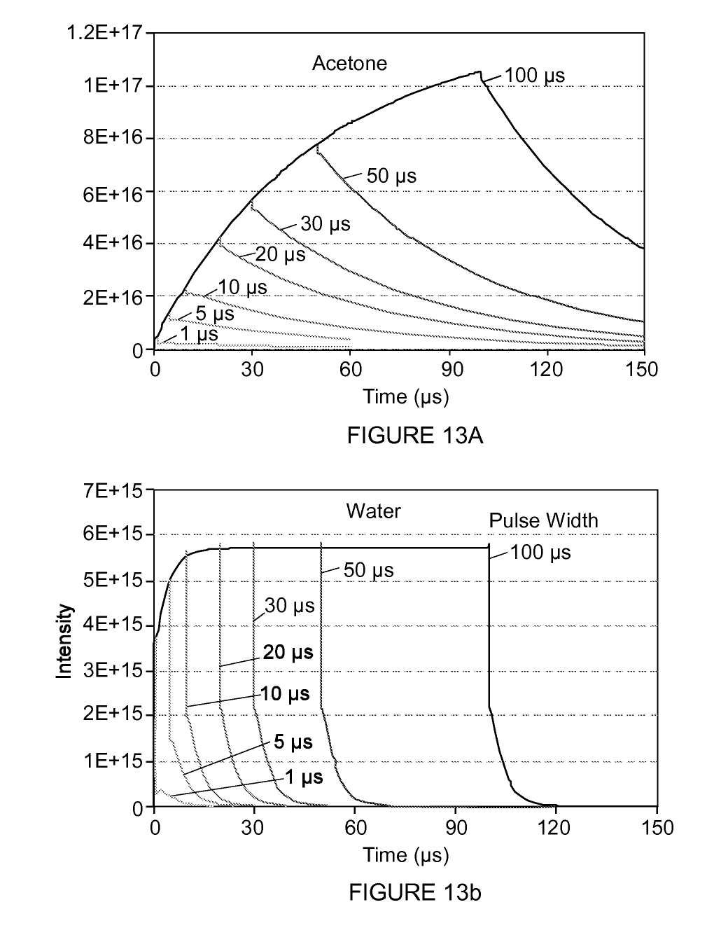

FIG. 12 shows methods for spectrally isolating the singlet oxygen optical emission from potential interferences such as PS fluorescence and tissue autofluorescence. Data was obtained in vitro in different solutions. FIG. 12A shows a three filter approach, indicated with the vertical bars. FIG. 12B shows a multiple wavelength approach, which includes measuring a signal at multiple wavelength points, curve fitting (non-linear expression) for out-of-band features, and subtracting calculated out-of-band components from in-band signal of singlet O₂.

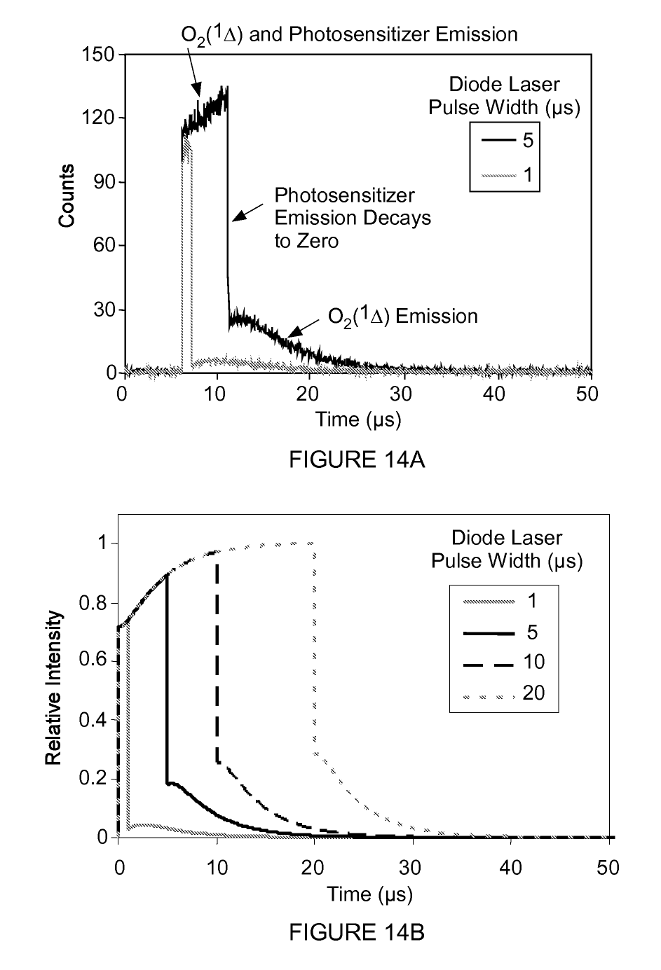

FIG. 13 shows optimizing the singlet oxygen emission using appropriate excitation pulselengths. For example, FIG. 13A shows predictions for the optical signals due to both the PS and singlet oxygen produced in an acetone solvent. Acetone is a weak quencher of singlet oxygen and the signal observed is predominantly due to singlet oxygen. The seven curves show the production of singlet oxygen for a variety of excitation pulses ranging from 1 to 100 μs. The amount of singlet oxygen produced rises as the pulses become longer. In FIG. 13B, the solvent is water, a much stronger quencher of singlet oxygen. In this case the overall amount of singlet oxygen produced is less and the pulseshape is distinct from the acetone (weak quenching case). For these data the singlet oxygen during and after the pulse is still visible, but the effects of quenching are clear. It appears for water that the maximum concentration of singlet oxygen is reached for a pulse of about 10 μs. This strategy can maximize the singlet oxygen in tissue.

Real-time dosimetry can be provided by simultaneous monitoring of singlet O₂ and PS fluorescence during PDT treatment. PDT efficacy can be improved by monitoring PDT treatment in real-time. The response parameters can be integrated into the PDT treatment in real-time by tailoring the treatment process by adjusting the various operating parameters of the light source. A PDT treatment can be modulated with wavelength selection, pulsed or CW light source, pulse width, duty cycle (the relation between repetition rate and pulse-width) and light source average power. Optimizing a treatment process can reduce the treatment time per session, and how many treatments must be performed. If too much light is delivered, the drug can photobleach and the tissue can de-oxygenate. With real-time monitoring, a clinician or physician knows to lower the energy or fluence delivered. If singlet oxygen is below a threshold value, then the light source energy or fluence can be increased.

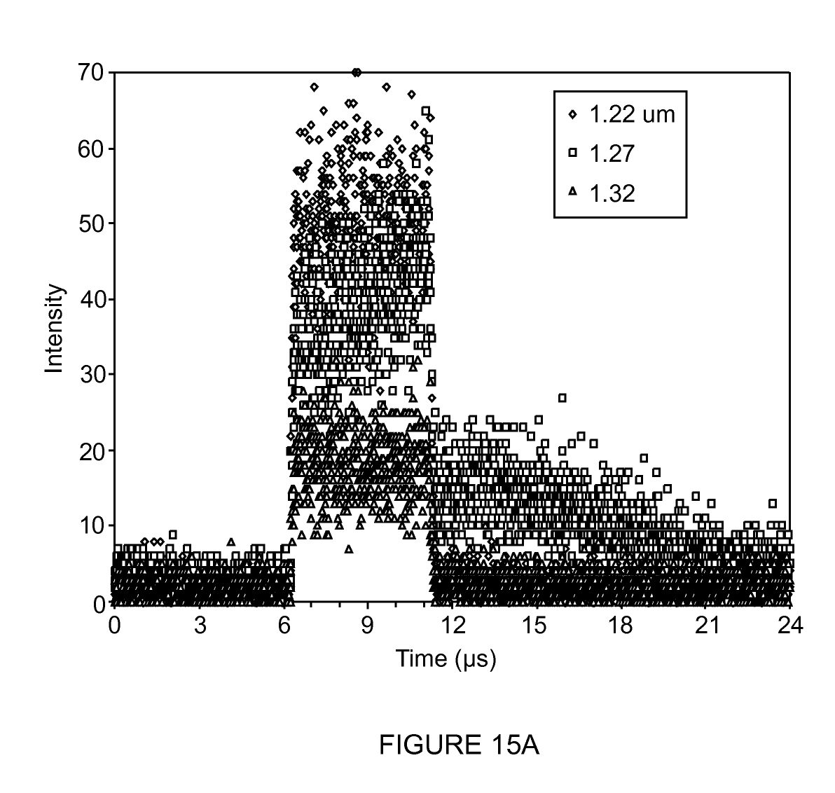

FIG. 14 shows that the PDT treatment response can be optimized by adjusting singlet O2 production. FIG. 14A shows experimental results, while FIG. 14B shows predictions. Singlet O₂ production during diode laser pulse ON varies depending on pulse width. A combination of duty cycle and pulse width can be used to optimize singlet O₂ production. FIG. 14B shows a modeled PDT mechanism to optimize singlet O₂ signal.

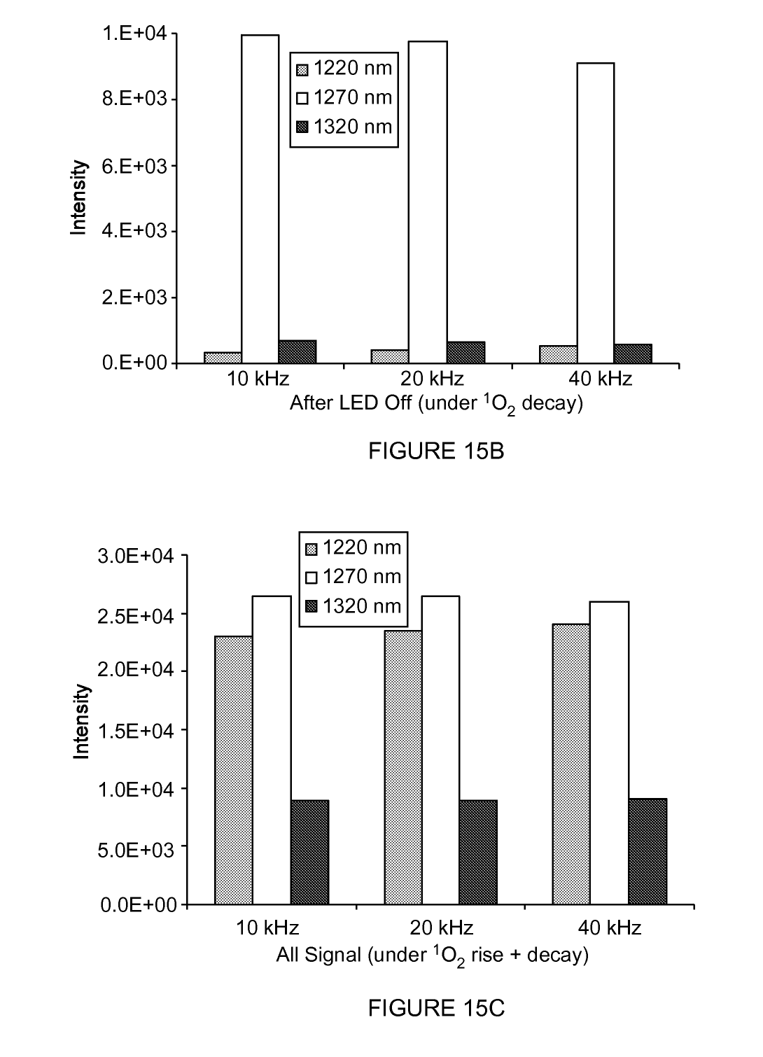

FIG. 15A shows in vitro results with an LED as the excitation source. A pulsed LED can be used to produce singlet oxygen, and the time resolved data acquisition and reduction methods of the technology can be used. The PS was PPiX in water. The LED pulsewidth was 5 micro seconds at 40 kHz. The signals of the PS at 1.22 and 1.32 microns are shown as is the singlet oxygen (much longer lived). FIGS. 15B and 15C show the reduced data. FIG. 15B shows data extracted only after the LED is off at each pulse. FIG. 15C shows data extracted from the period with the LED on.

The above-described techniques can be implemented in digital electronic circuitry, or in computer hardware, firmware, software, or in combinations of them. The implementation can be as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Method steps can be performed by one or more programmable processors executing a computer program to perform functions of the technology by operating on input data and generating output. Method steps can also be performed by, and apparatus can be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit), or the like. Subroutines can refer to portions of the stored computer program and/or the processor, and/or the special circuitry that implement one or more functions.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

The terms “module” and “function,” as used herein, mean, but are not limited to, a software or hardware component which performs certain tasks. A module may advantageously be configured to reside on addressable storage medium and configured to execute on one or more processors. A module may be fully or partially implemented with a general purpose integrated circuit (IC), DSP, FPGA or ASIC. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules may be combined into fewer components and modules or further separated into additional components and modules. Additionally, the components and modules may advantageously be implemented on many different platforms, including computers, computer servers, data communications infrastructure equipment such as application-enabled switches or routers, or telecommunications infrastructure equipment, such as public or private telephone switches or private branch exchanges (PBX). In any of these cases, implementation may be achieved either by writing applications that are native to the chosen platform, or by interfacing the platform to one or more external application engines.

To provide for interaction with a user, the above described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The above described techniques can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an example implementation, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet, and include both wired and wireless networks. Communication networks can also all or a portion of the PSTN, for example, a portion owned by a specific carrier.

The computing system can include clients and servers. A client and server are generally remote from each other and typically interact through a communication network. The relationship of client and server arises by virtue of computer programs running on the respective computers and having a client-server relationship to each other.

While the invention has been particularly shown and described with reference to specific illustrative embodiments, it should be understood that various changes in form and detail may be made without departing from the spirit and scope of the invention.

1. A method for monitoring singlet oxygen during photodynamic therapy (PDT), comprising:
directing excitation light to a photosensitizer in a target region;
receiving light emitted by the photosensitizer and/or singlet oxygen generated in the target region;
filtering the returning light to isolate emission from the singlet oxygen;
monitoring the emission from the singlet oxygen; and
determining a concentration of the singlet oxygen based on an emission signal measured by a detection system.

2. The method of claim 1 further comprising monitoring the photosensitizer and/or singlet oxygen between pulses of the excitation light.

3. The method of claim 1 further comprising monitoring the photosensitizer and/or singlet oxygen when a source of the excitation light is on.

4. The method of claim 1 further comprising monitoring the emission from the singlet oxygen using a detection system including a photomultiplier tube, an avalanche photodiode, an array of avalanche photodiodes, a CCD camera or a near infrared camera.

5. The method of claim 1 further comprising monitoring the emission from the singlet oxygen at 1.27 micron.

6. The method of claim 1 further comprising generating the excitation light using a pulsed source, a continuous source, a low power, pulsed diode laser or a light emitting diode.

7. The method of claim 1 wherein the PDT is for treatment of acne.

8. The method of claim 1 wherein the PDT is for treatment of cancer.

9. An apparatus for photodynamic therapy (PDT), comprising:
a light source configured to provide excitation light for a photosensitizer;
an optical system configured to direct the excitation light to a target region and receive light emitted by the photosensitizer and/or singlet oxygen generated in the target region;
a detection system configured to receive the light emitted by the photosensitizer and/or the singlet oxygen;
a filter system configured to spectrally discriminate between emission from the photosensitizer and the singlet oxygen; and
a processor configured to determine concentrations of the singlet oxygen and/or the photosensitizer based on an emission signal measured by the detection system.

10. The apparatus of claim 9 wherein the detection system is configured to measure the photosensitizer and/or singlet oxygen between pulses of the excitation light from the light source.

11. The apparatus of claim 9 wherein the detection system is configured to measure the photosensitizer and/or singlet oxygen when the light source is on.

12. The apparatus of claim 9 wherein the processor is configured to provide feedback control to the light source for a PDT treatment.

13. The apparatus of claim 9 wherein the detection system includes a photomultiplier tube, an avalanche photodiode, an array of avalanche photodiodes, a CCD camera or a near infrared camera.

14. The apparatus of claim 9 wherein the detection system is adapted to detect 1.27 micron radiation from singlet oxygen emission.

15. The apparatus of claim 9 wherein the light source is a pulsed source, a continuous source, a low power, pulsed diode laser or an LED.

16. The apparatus of claim 9 further comprising a dichroic beamsplitter configured to pass the excitation light from the pulsed light source to an optical waveguide and redirect the light returning from the optical waveguide to the detection system.

17. The apparatus of claim 9 wherein the PDT is for treatment of acne.

18. The apparatus of claim 9 wherein the PDT is for treatment of cancer.

19. An apparatus for photodynamic therapy (PDT), comprising:
means for directing excitation light to a photosensitizer in a target region;
means for receiving light emitted by the photosensitizer and/or singlet oxygen generated in the target region;
means for filtering the returning light to isolate emission from the singlet oxygen;
means for monitoring the emission from the singlet oxygen; and
means for determining a concentration of the singlet oxygen based on an emission signal measured by a detection system.
